(19) United States
(12) Patent Application Publication
(10) Pub. No.: US 2025/0256074 A1
(43) Pub. Date: Aug. 14, 2025

(54) CURVED OUTER SHEATH FOR A SHUNT DEVICE DELIVERY DEVICE

(71) Applicant: EDWARDS LIFESCIENCES CORPORATION, Irvine, CA (US)

(72) Inventors: Scott Louis Pool, Laguna Hills, CA (US); Molly Bettencourt, Huntington Beach, CA (US)

(73) Assignee: EDWARDS LIFESCIENCES CORPORATION, Irvine, CA (US)

(21) Appl. No.: 19/098,216

(22) Filed: Apr. 2, 2025

Related U.S. Application Data
(63) Continuation of application No. PCT/US2023/034388, filed on Oct. 3, 2023.
(60) Provisional application No. 63/378,174, filed on Oct. 3, 2022.

(51) Int. Cl. A61M 27/00

(52) U.S. Cl. CPC A61M 27/002; A61M 2210/125

(57) ABSTRACT

An outer sheath for surrounding and covering components of a delivery device includes a straight section including an aperture extending fully through the straight section. The outer sheath further includes a pre-curved section extending distally from an end of the straight section and including an aperture extending fully through the pre-curved section. The straight section and the pre-curved section include a varying stiffness along a length of the straight section and the pre-curved section.

### CROSS-REFERENCE TO RELATED APPLICATION(S)

This application is a continuation of International Application No. PCT/US2023/034388, filed Oct. 3, 2023, which claims the benefit of U.S. Provisional Application No. 63/378,174, filed Oct. 3, 2022, the disclosures of which are hereby incorporated by reference in their entireties.

### BACKGROUND

The present disclosure relates generally to implantable devices for a human body, and more particularly to an outer sheath for a delivery device used to aid in delivering and placing a shunt device within a human body.

Shunt devices can be positioned in the heart to shunt blood between the left atrium and the right atrium to reduce pressure in the left atrium. The left atrium can experience elevated pressure due to abnormal heart conditions caused by age and/or disease. For example, shunt devices can be used to treat patients with heart failure (also known as congestive heart failure). Shunt device can be positioned in the septal wall between the left atrium and the right atrium to shunt blood from the left atrium into the right atrium, thus reducing the pressure in the left atrium.

### SUMMARY

An outer sheath for surrounding and covering components of a delivery device includes a straight section including an aperture extending fully through the straight section. The outer sheath further includes a pre-curved section extending distally from an end of the straight section and including an aperture extending fully through the pre-curved section. The straight section and the pre-curved section include a varying stiffness along a length of the straight section and the pre-curved section.

### BRIEF DESCRIPTION OF THE DRAWINGS

### Anatomy of Heart H and Vasculature V

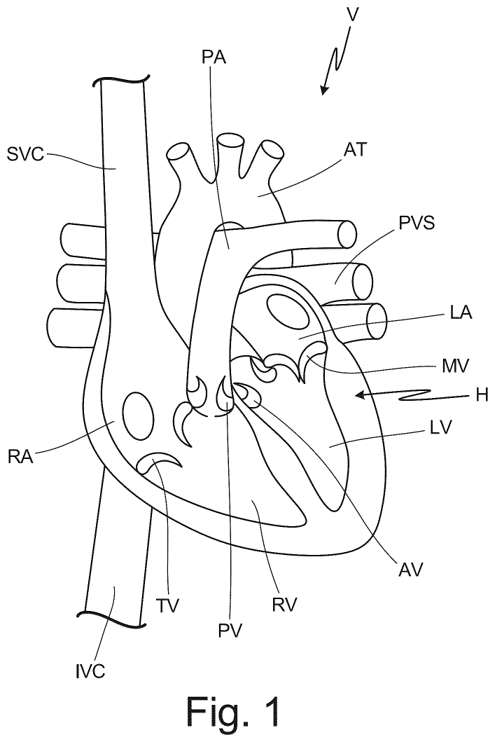

FIG. 1 is a schematic diagram of a heart and vasculature.

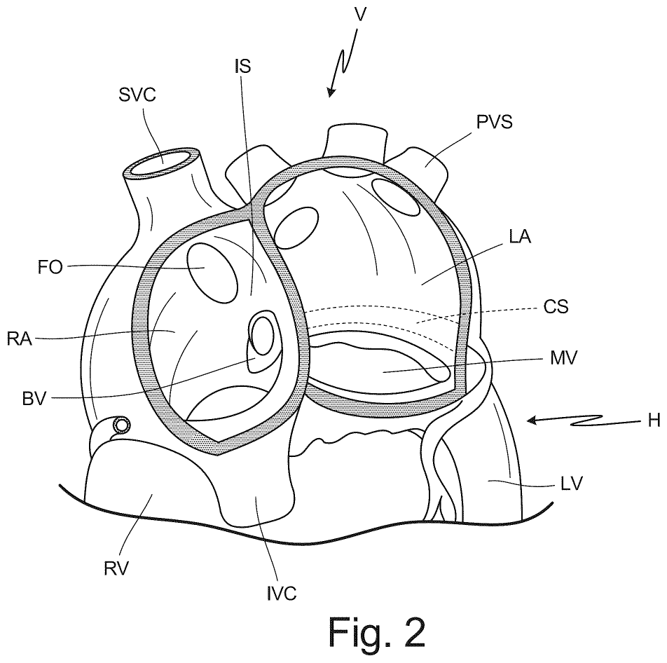

FIG. 2 is a schematic cross-sectional view of the heart.

### Shunt Devices 100 and 100′

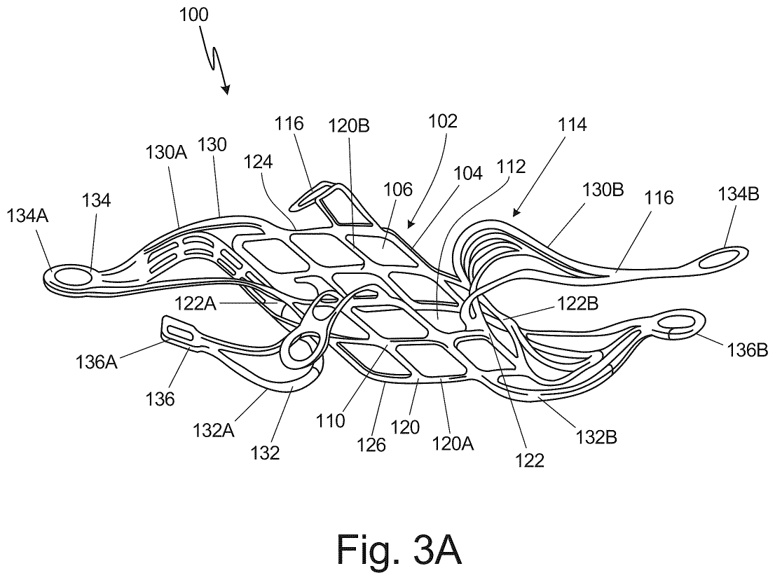

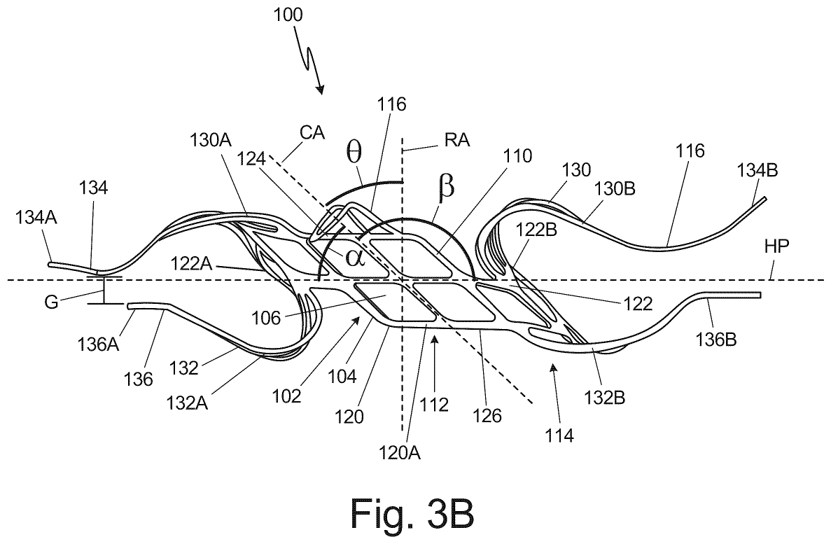

FIG. 3A is a perspective view of a shunt device.

FIG. 3B is a side view of the shunt device.

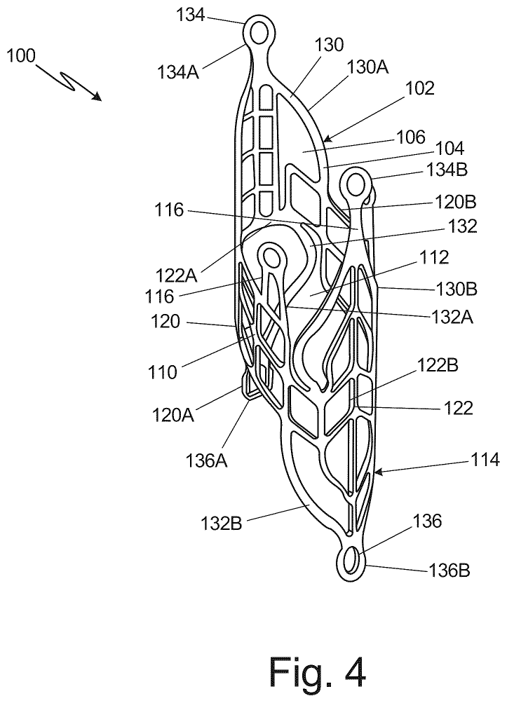

FIG. 4 is a perspective view of the shunt device in a collapsed configuration.

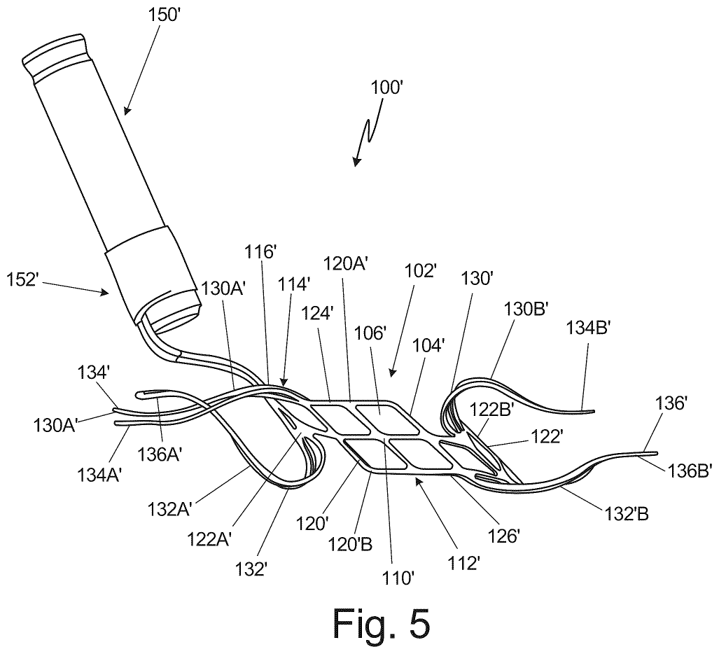

FIG. 5 is a perspective view of a shunt device including a sensor.

### Delivery Catheter 200

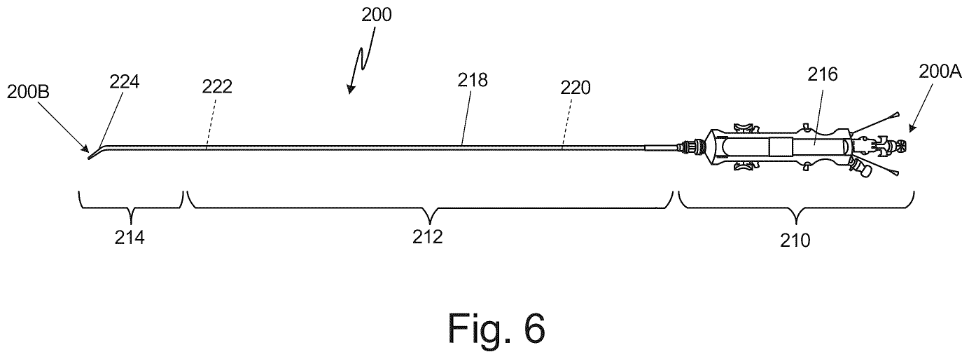

FIG. 6 is a side view of a delivery catheter.

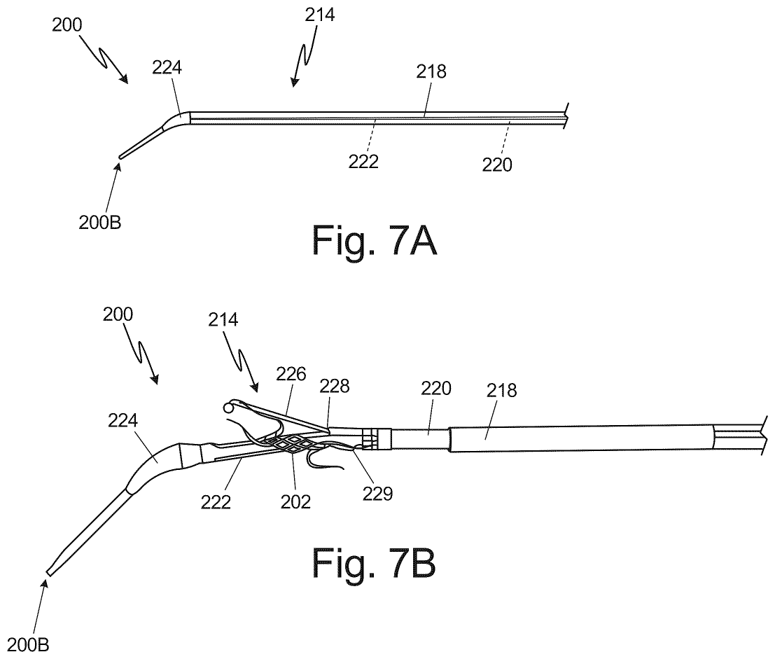

FIG. 7A is a side view of a distal portion of the delivery catheter in a sheathed state.

FIG. 7B is a side view of the distal portion of the delivery catheter in an unsheathed state.

### Delivery Method 300

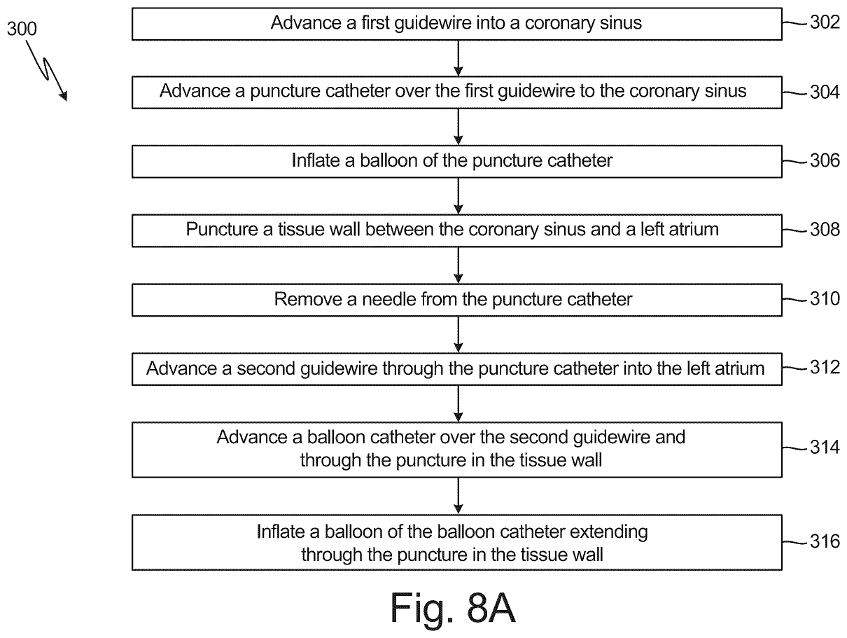

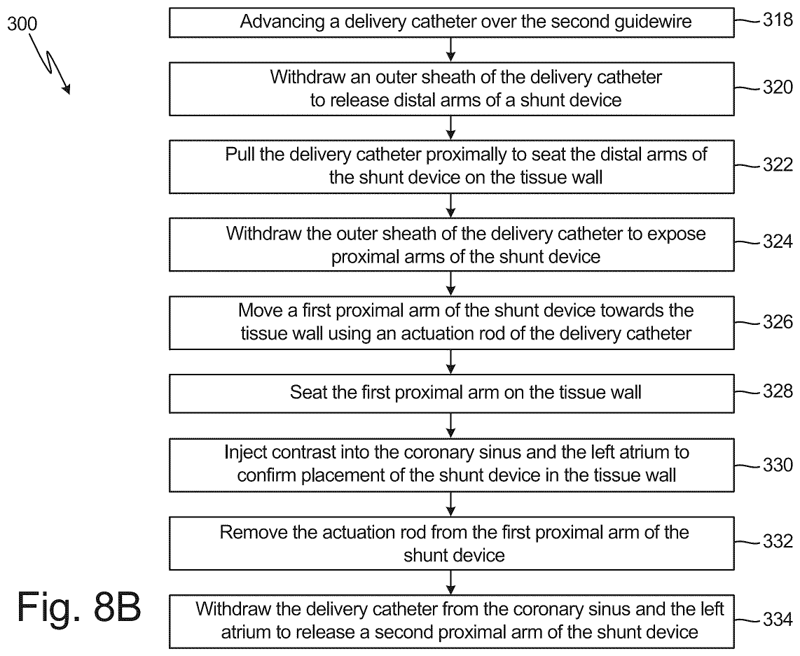

FIG. 8A is a flow chart showing steps for creating a puncture in a tissue wall between a coronary sinus and a left atrium.

FIG. 8B is a flow chart showing steps for implanting a shunt device in the tissue wall between the coronary sinus and the left atrium.

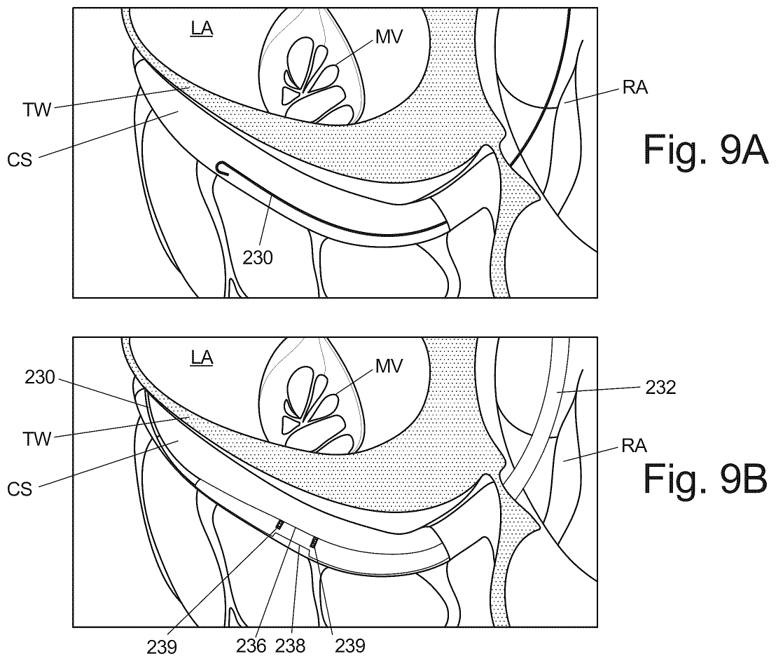

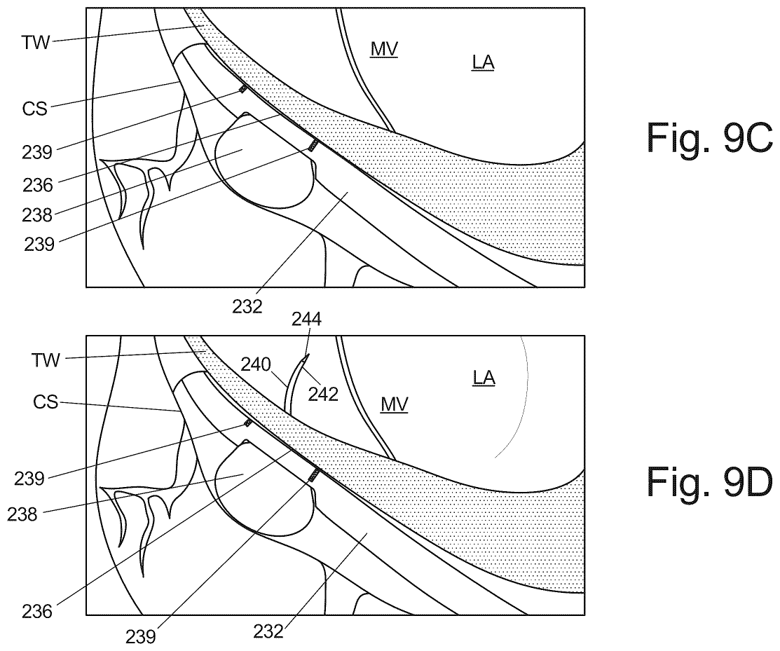

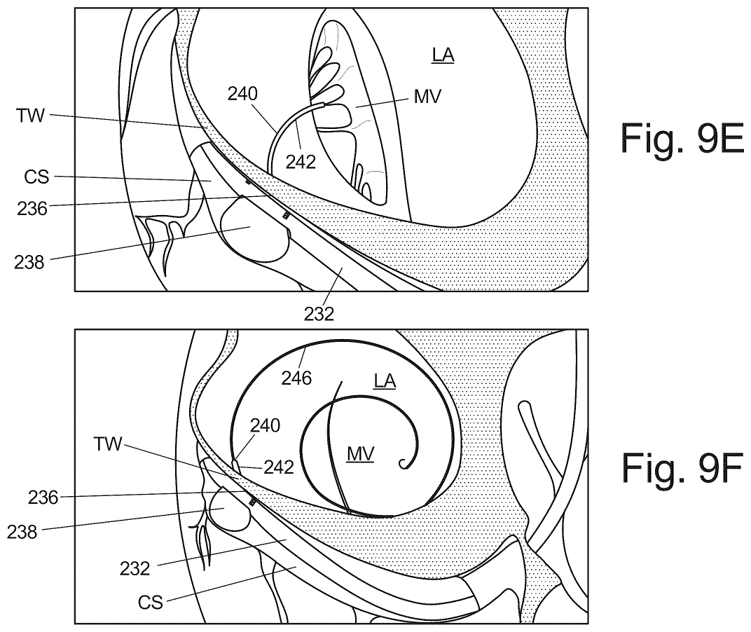

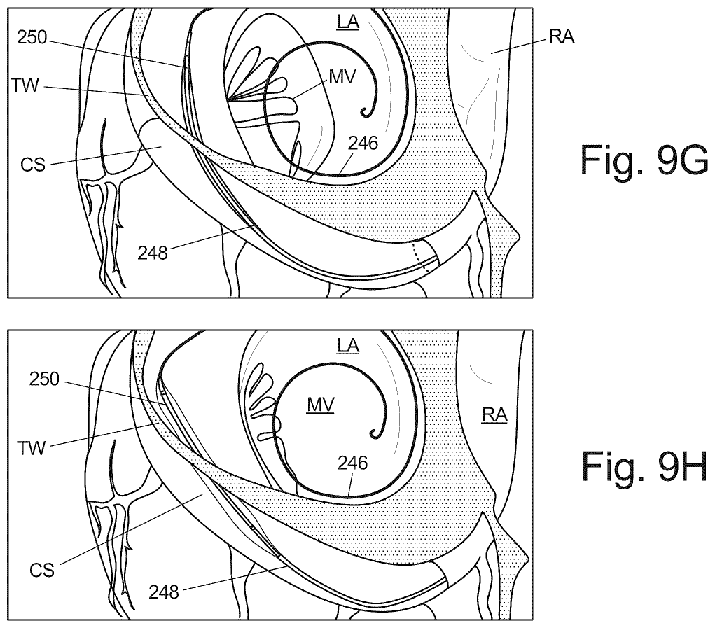

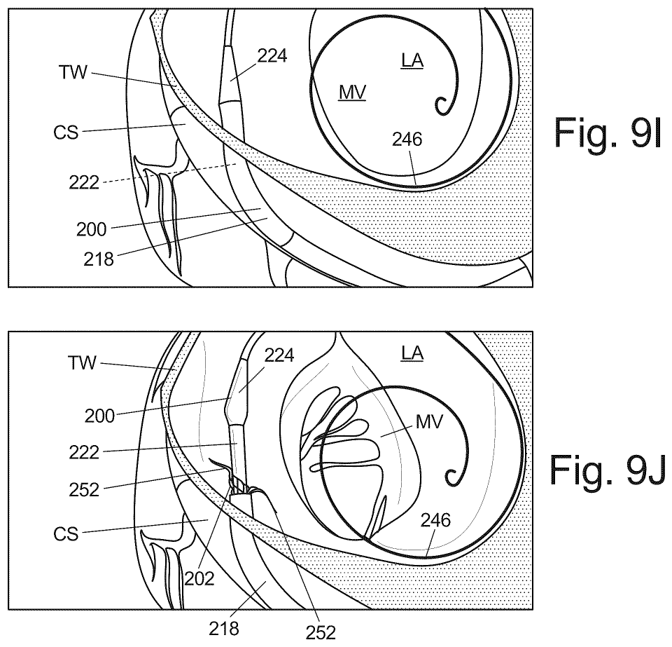

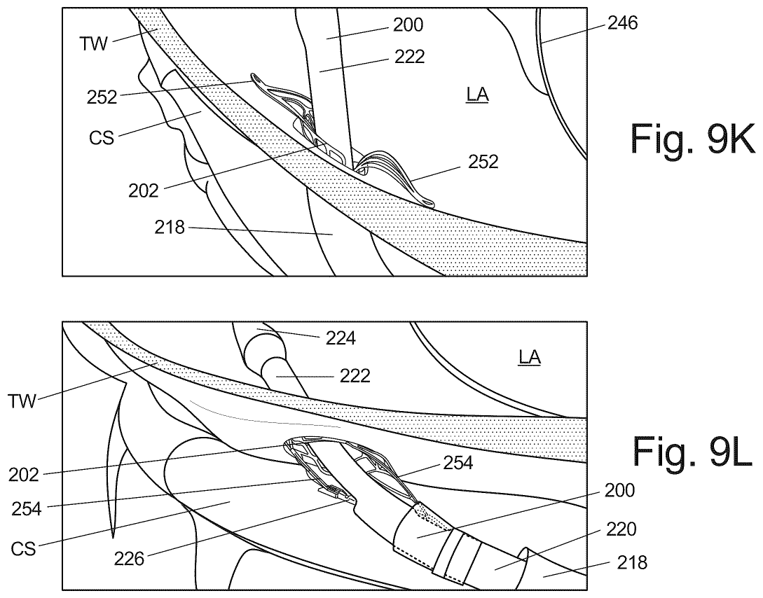

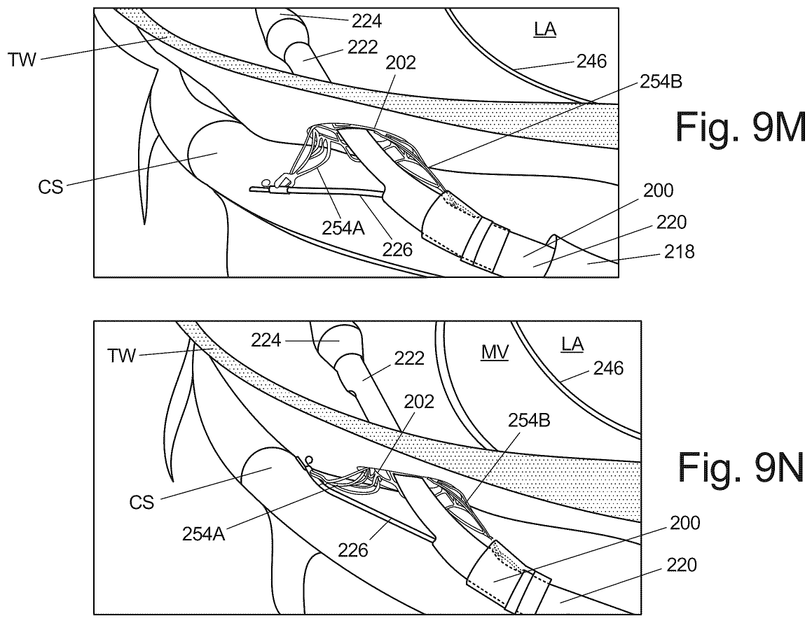

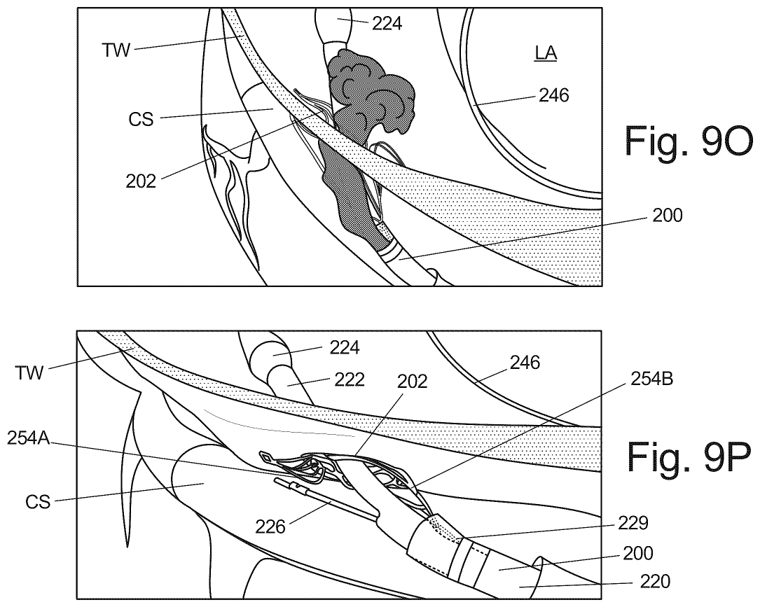

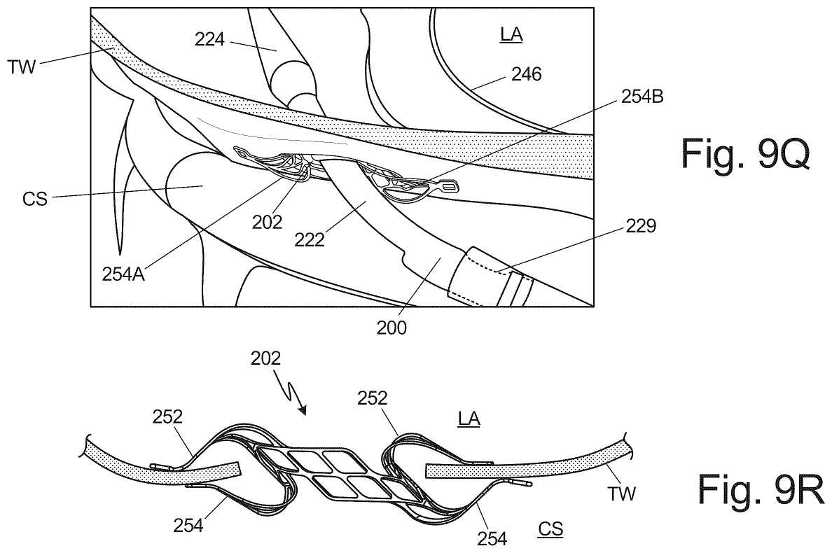

FIGS. 9A-9R are schematic views showing the steps for implanting a shunt device in the tissue wall between the coronary sinus and the left atrium.

### Delivery Device 1000

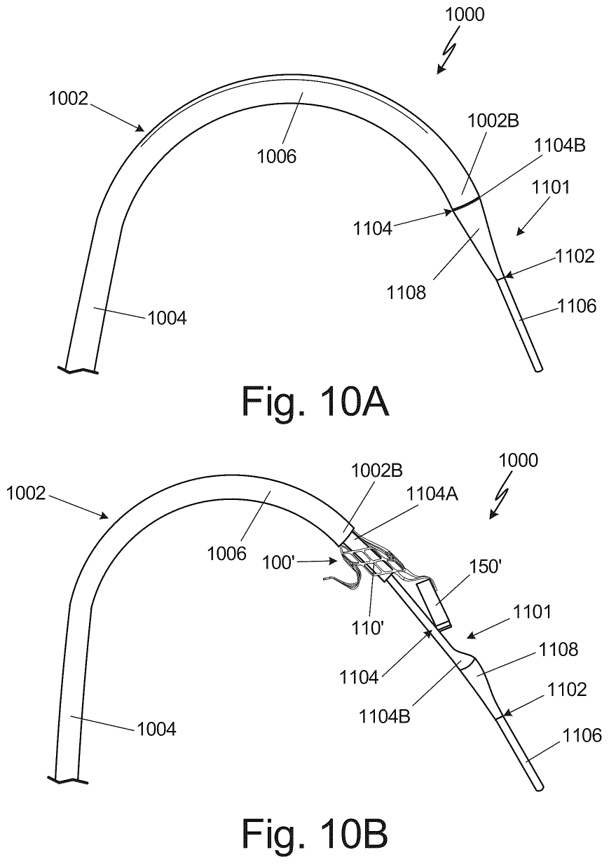

FIG. 10A is a perspective view of a delivery device including an outer sheath.

FIG. 10B is a perspective view of the delivery device with the outer sheath pulled back to expose a shunt device.

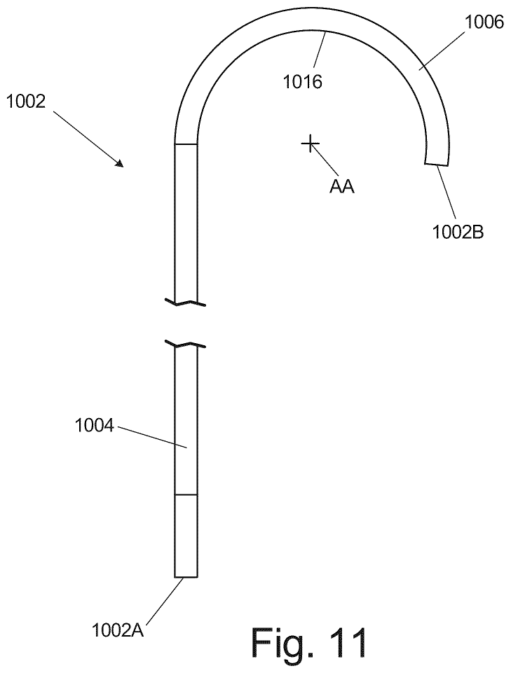

FIG. 11 is a side view of the outer sheath used in the delivery device.

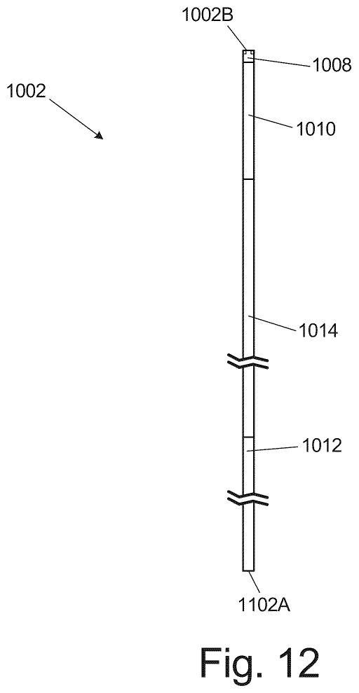

FIG. 12 is a side view of the outer sheath in a straight configuration.

### DETAILED DESCRIPTION

### Anatomy of Heart H and Vasculature V (FIGS. 1-2)

FIG. 1 is a schematic diagram of heart H and vasculature V. FIG. 2 is a cross-sectional view of heart H. FIGS. 1-2 will be described together. FIGS. 1-2 show heart H, vasculature V, right atrium RA, right ventricle RV, left atrium LA, left ventricle LV, superior vena cava SVC, inferior vena cava IVC, tricuspid valve TV (shown in FIG. 1), pulmonary valve PV (shown in FIG. 1), pulmonary artery PA (shown in FIG. 1), pulmonary veins PVS, mitral valve MV, aortic valve AV (shown in FIG. 1), aorta AT (shown in FIG. 1), coronary sinus CS (shown in FIG. 2), thebesian valve BV (shown in FIG. 2), inter-atrial septum IS (shown in FIG. 2), and fossa ovalis FO (shown in FIG. 2).

Heart H is a human heart that receives blood from and delivers blood to vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC and inferior vena cava IVC. Right atrium RA pumps the blood through tricuspid valve TV into right ventricle RV. The blood is then pumped by right ventricle RV through pulmonary valve PV into pulmonary artery PA. The blood flows from pulmonary artery PA into arteries that delivery the deoxygenated blood to the lungs via the pulmonary circulatory system. The lungs can then oxygenate the blood.

The left side of heart H, including left atrium LA and left ventricle LV, receives the oxygenated blood from the lungs and pumps the blood to the body. Blood flows into left atrium LA from pulmonary veins PVS. Left atrium LA pumps the blood through mitral valve MV into left ventricle LV. The blood is then pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 2. Coronary sinus CS has an increasing diameter as it connects to right atrium RA.

Inter-atrial septum IS and fossa ovalis FS are also shown in FIG. 2. Inter-atrial septum IS is the wall that separates right atrium RA from left atrium LA. Fossa ovalis FS is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FS. The foramen ovale serves as a functional shunt in utero, allowing blood to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and the ductus venosus. The oxygenated blood moves through inferior vena cava IVC to right atrium RA. The opening of inferior vena cava IVC in right atrium RA is positioned to direct the oxygenated blood through right atrium RA and the foramen ovale into left atrium LA. Left atrium LA can then pump the oxygenated blood into left ventricle LV, which pumps the oxygenated blood to aorta AT and the systemic circulatory system. This allows the pulmonary circulatory system to be bypassed in utero. Upon birth, respiration expands the lungs, blood begins to circulate through the lungs to be oxygenated, and the foramen ovale closes to form fossa ovalis FS.

Shunt devices can be positioned in heart H to shunt blood between left atrium LA and right atrium RA. Left atrium LA can experience elevated pressure due to abnormal heart conditions. It has been hypothesized that patients with elevated pressure in left atrium LA may benefit from a reduction of pressure in left atrium LA. Shunt devices can be used in these patients to shunt blood from left atrium LA to right atrium RA to reduce the pressure of blood in left atrium LA, which reduces the systolic preload on left ventricle LV. Reducing pressure in left atrium LA further relieves back-pressure on the pulmonary circulation to reduce the risk of pulmonary edema.

For example, shunt devices can be used to treat patients with heart failure (also known as congestive heart failure). The hearts of patients with heart failure do not pump blood as well as they should. Heart failure can affect the right side and/or the left side of the heart. Diastolic heart failure (also known as heart failure with preserved ejection fraction) refers to heart failure occurring when the left ventricle is stiff (having less compliance), which makes it hard to relax appropriately and fill with blood. This leads to increased end-diastolic pressure, which causes an elevation of pressure in left atrium LA. There are very few, if any, effective treatments available for diastolic heart failure. Other examples of abnormal heart conditions that cause elevated pressure in left atrium LA are systolic dysfunction of the left ventricle and valve disease.

Septal shunt devices (also called inter-atrial shunt devices) are positioned in inter-atrial septum IS to shunt blood directly from left atrium LA to right atrium RA. Typically, septal shunt devices are positioned in fossa ovalis FS, as fossa ovalis FS is a thinner area of tissue in inter-atrial septum IS where the two atria share a common wall. If the pressure in right atrium RA exceeds the pressure in left atrium LA, septal shunt devices can allow blood to flow from right atrium RA to left atrium LA. This causes a risk of paradoxical stroke (also known as paradoxical embolism), as emboli can move from right atrium RA to left atrium LA and then into aorta AT and the systemic circulation.

Shunt devices can also be left atrium to coronary sinus shunt devices that are positioned in a tissue wall between left atrium LA and coronary sinus CS where the two structures are in close approximation. Left atrium to coronary sinus shunt devices move blood from left atrium LA into coronary sinus CS, which then delivers the blood to right atrium RA via thebesian valve BV, the natural orifice of coronary sinus CS. Coronary sinus CS acts as an additional compliance chamber when using a left atrium to coronary sinus shunt device. Left atrium to coronary sinus shunt devices further provide increased protections against paradoxical strokes, as the blood would have to flow retrograde from right atrium RA through coronary sinus CS before entering left atrium LA. Further, left atrium to coronary sinus shunt devices also provide protection against significant right atrium RA to left atrium LA shunting, as again the blood would have to flow retrograde from right atrium RA through coronary sinus CS before entering left atrium LA.

### Shunt Devices 100 and 100′ (FIGS. 3A-5)

FIG. 3A is a perspective view of shunt device 100. FIG. 3B is a side view of shunt device 100. FIG. 4 is a perspective view of shunt device 100 in a collapsed configuration. FIGS. 3A, 3B, and 4 will be described together. Shunt device 100 includes body 102, which is formed of struts 104 and openings 106. Body 102 includes central flow tube 110, flow path 112, and arms 114. Shunt device 100 also includes tissue capture features 116. Central flow tube 110 has side portions 120 (including side portion 120A and side portion 120B), end portions 122 (including end portion 122A and end portion 122B), first axial end 124, and second axial end 126. Arms 114 include distal arms 130 (including distal arm 130A and distal arm 130B) and proximal arms 132 (including proximal arm 132A and proximal arm 132B). Distal arms 130 have terminal ends 134 (including terminal end 134A and terminal end 134B). Proximal arms 132 have terminal ends 136 (including terminal end 136A and terminal end 136B). FIG. 3B further shows gap G, horizontal reference plane HP, perpendicular reference axis RA, central axis CA, tilt angle θ, first angle α, and second angle β.

Shunt device 100 is a cardiovascular shunt. Shunt device 100 is shown in an expanded configuration in FIGS. 3A-3B. Shunt device 100 is formed of a super-elastic material that is capable of being compressed into a catheter for delivery into the body that can then retain its relaxed, or expanded, shape when it is released from the catheter. For example, shunt device 100 can be formed of a shape-memory material, such as nitinol (a nickel titanium alloy). Shunt device 100 is shown in a compressed configuration in FIG. 4. Upon delivery into the body, shunt device 100 will expand back to its relaxed, or expanded, shape. Shunt device 100 can be sterilized before being delivered into the body. Shunt device 100 has body 102 that is formed of interconnected struts 104. Openings 106 in body 102 are defined by struts 104. Body 102 of shunt device 100 is formed of struts 104 to increase the flexibility of shunt device 100 to enable it to be compressed and expanded.

Body 102 includes central flow tube 110 that forms a center portion of shunt device 100. Central flow tube 110 is tubular in cross-section but is formed of struts 104 and openings 106. Central flow tube 110 can be positioned in a puncture or opening in a tissue wall and hold the puncture open. Flow path 112 is an opening extending through central flow tube 110. Flow path 112 is the path through which blood flows through shunt device 100 when shunt device 100 is implanted in the body. Arms 114 extend from central flow tube 110. Arms 114 extend outward from central flow tube 110 when shunt device 100 is in an expanded configuration. Arms 114 hold shunt device 100 in position in the tissue wall when shunt device 100 is implanted in the body.

When shunt device 100 is implanted in the tissue wall between the left atrium and the coronary sinus of the heart, central flow tube 110 holds the puncture open so blood can flow from the left atrium to the coronary sinus through flow path 112. Struts 104 of central flow tube 110 form a lattice or cage of sorts that is sufficient to hold the puncture in the tissue wall open around central flow tube 110. Central flow tube 110 extends from first axial end 124 to second axial end 126. Central flow tube 110 is designed to have an axial length, as measured from first axial end 124 to second axial end 126, that approximates the thickness of the tissue wall between the left atrium and the coronary sinus. When shunt device 100 is implanted in the tissue wall between the left atrium and the coronary sinus, first axial end 124 can be facing the left atrium (i.e., a left atrial side of shunt device 100) and second axial end 126 can be facing the coronary sinus (i.e., a coronary sinus side of shunt device 100). In other examples, the orientation of first axial end 124 and second axial end 126 can be reversed.

Central flow tube 110 has side portions 120 and end portions 122. Side portion 120A and side portion 120B form opposing sides of central flow tube 110. End portion 122A and end portion 122B form opposing ends of central flow tube 110. End portion 122A and end portion 122B each extend between and connect to side portion 120A and side portion 120B to form a generally circular or oval opening that defines flow path 112. Side portions 120 and end portions 122 form a tubular lattice for central flow tube 110. Struts 104 of central flow tube 110 define openings 106 in central flow tube 110. In some examples, openings 106 can be generally parallelogram-shaped. In other examples, openings 106 can be any regular or irregular shape as desired. For example, struts 104 of side portions 120 can form an array of parallelogram-shaped openings 106 in side portions 120. Struts 104 of end portions 122 can form openings 106 in end portions 122. Struts 104 of arms 114 can form openings 106 in arms 114.

As shown in FIG. 3B, central flow tube 110 is angled with respect to horizontal reference plane HP extending through shunt device 100. Horizontal reference plane HP lies generally in the plane of the tissue wall immediately adjacent to shunt device 100 when shunt device 100 is implanted in the tissue wall. End portions 122 are similarly angled with respect to horizontal reference plane HP. Perpendicular reference axis RA, as shown in FIG. 3B, is perpendicular to horizontal reference plane HP. As shown in FIG. 3B, central axis CA is an axis through the center of central flow tube 110 and flow path 112. Central axis CA extends through central flow tube 110 at tilt angle θ with respect to perpendicular reference axis RA. Accordingly, central axis CA defines the angle or tilt of central flow tube 110 with respect to perpendicular reference axis RA (and horizontal reference plane HP). End portions 122 of central flow tube 110 extend parallel to central axis CA.

Arms 114 of shunt device 100 include two distal arms 130 and two proximal arms 132. In some examples, individual ones of distal arms 130 and/or proximal arms 132 can be formed of multiple split arm portions. Arms 114 extend outward from end portions 122 of central flow tube 110 when shunt device 100 is in an expanded configuration. Distal arm 130A is connected to and extends away from end portion 122A, and distal arm 130B is connected to and extends away from end portion 122B. Proximal arm 132A is connected to and extends away from end portion 122A, and proximal arm 132B is connected to and extends away from end portion 122B. When shunt device 100 is implanted in the tissue wall between the left atrium and the coronary sinus, distal arms 130 will be positioned in the left atrium and proximal arms 132 will be positioned in the coronary sinus. Distal arms 130 each have terminal ends 134. Specifically, distal arm 130A has terminal end 134A, and distal arm 130B has terminal end 134B. Proximal arms 132 each have terminal ends 136. Specifically, proximal arm 132A has terminal end 136A, and proximal arm 132B has terminal end 136B.

Distal arms 130 and proximal arms 132 curl outward from end walls 122. As shown in FIG. 3B, each of distal arms 130 and proximal arms 132 has a proximal portion adjacent to central flow tube 110 that forms a shallow curve or arc in a direction away from end walls 122 of central flow tube 110. Each of distal arms 130 and proximal arms 132 flattens out towards respective terminal ends 134 and 136 such that a portion of each of distal arms 130 and proximal arms 132 at or adjacent to the respective terminal end 134 or 136 is generally parallel to horizontal reference plane HP. Accordingly, an axis drawn through terminal end 134A and an axis drawn through terminal end 136B, which are approximated in FIG. 3B as axes in the plane of horizontal reference plane HP for simplicity, can each form first angle α with central axis CA through central flow tube 110. Similarly, an axis drawn through terminal end 134B, and an axis drawn through terminal end 136A, which are approximated in FIG. 3B as axes in the plane of horizontal reference plane HP for simplicity, can each form second angle β with central axis CA through central flow tube 110. Alternatively, distal arms 130 and proximal arms 132 do not flatten out and become parallel to horizontal reference plane HP but instead approach horizontal reference plane HP at an angle and/or have respective terminal ends 134 and 136 that angle away from horizontal reference plane HP. In such examples, first angle α and second angle β are approximations of the central angle for the arcs from end walls 122 to the tissue wall that each respective arm encompasses when shunt device 100 is implanted in the tissue wall. Put more simply, first angle α is the angle between central axis CA and horizontal reference plane HP, and second angle β is the supplementary angle to first angle α. In some examples, first angle α can be less than ninety degrees (<90°) and second angle β can be greater than ninety degrees (>90°). In other examples, first angle α and second angle β can be any suitable combination of angles that add to one hundred eighty degrees (180°). The difference between first angle α and second angle β (and the corresponding curvature of ones of distal arms 130 and proximal arms 132) accommodates for the tilt of central flow tube 110.

As shown in FIG. 3B, distal arm 130A and distal arm 130B extend outwards from central flow tube 110 in opposite directions parallel to horizontal reference plane HP. Distal arm 130A and distal arm 130B can be aligned with each other (i.e., oriented at 180° to each other across central flow tube 110). In some examples, distal arm 130A has a longer length than distal arm 130B. In other examples, distal arm 130A has a shorter length than distal arm 130B. In yet other examples, distal arms 130 can have similar lengths. Proximal arm 132A and proximal arm 132B extend outwards from central flow tube 110 in opposite directions parallel to horizontal reference plane HP. Proximal arm 132A and proximal arm 132B can be aligned with each other (i.e., oriented at 180° to each other across central flow tube 110). In some examples, proximal arm 132A has a shorter length than proximal arm 132B. In other examples, proximal arm 132A has a longer length than proximal arm 132B. In yet other examples, proximal arms 132 can have similar lengths. In some examples, distal arm 130A has generally the same length and shape as proximal arm 132B, and distal arm 130B has generally the same length and shape as proximal arm 132A. In other examples, each of distal arms 130 and proximal arms 132 can have different lengths and shapes, though the overall shape of each arm is similar. As such, shunt device 100 has some degree of inverse symmetry across horizontal reference plane HP, as shown in FIG. 3B.

Shunt device 100 is generally elongated longitudinally but is relatively narrow laterally. Stated another way, distal arms 130 and proximal arms 132 are not annular or circular, but rather extend outward generally in only one plane. As shown in FIG. 3B, shunt device 100 has a generally H-shape when viewing a side of shunt device 100. The elongated shape of shunt device 100 means that when compressed it elongates along a line, as shown in FIG. 4, so as to better fit within a catheter.

Terminal ends 134 of distal arms 130 and terminal ends 136 of proximal arms 132 converge towards one another. Distal arms 130 and proximal arms 132 form two pairs of arms. That is, each of distal arms 130 forms a clamping pair with a corresponding one of proximal arms 132. Distal arm 130A and proximal arm 132A form a first pair of arms extending outward from a first side of central flow tube 110, and terminal end 134A of distal arm 130A converges towards terminal end 136A of proximal arm 132A. Distal arm 130B and proximal arm 132B form a second pair of arms extending outward from a second side of central flow tube 110, and terminal end 134B of distal arm 130B converges towards terminal end 136B of proximal arm 132B. Gap G between terminal ends 134 and terminal ends 136 is sized to be slightly smaller than an approximate thickness of the tissue wall between the left atrium and the coronary sinus, or another tissue wall of interest. This allows distal arms 130 and proximal arms 132 to flex outwards and grip the tissue wall when implanted to help hold shunt device 100 in place against the tissue wall. Thus, a distance corresponding to gap G, as measured once shunt device 100 is implanted, may be slightly different between different clamping pairs of distal arms 130 and proximal arms 132 depending on anatomical variations along the particular tissue wall. Terminal ends 134 of distal arms 130 and terminal ends 136 of proximal arms 132 can also have openings or indentations that are configured to engage a delivery tool to facilitate implantation of shunt device 100, for example actuating rods of a delivery tool. Additionally, terminal ends 134 of distal arms 130 and terminal ends of proximal arms 132 can include locations for radiopaque markers to permit visualization of the positioning of shunt device 100.

When implanted in the tissue wall, distal arms 130 and proximal arms 132 are designed such that the projection of distal arms 130 and proximal arms 132 into the left atrium and the coronary sinus, respectively, is minimized. This minimizes the disruption of the natural flow patterns in the left atrium and the coronary sinus. Shunt device 100 can also be designed so that the profile of proximal arms 132 projecting into the coronary sinus is lower than the profile of distal arms 130 projecting into the left atrium to minimize disruption of the natural blood flow through the coronary sinus and to reduce the potential for proximal arms 132 to block the narrower passage of the coronary sinus.

Tissue capture features 116 can take several different forms. For example, tissue capture features 116 connected to central flow tube 110 at first axial end 124 and/or second axial end 126 can be tabs that extend outward from side portions 120. Tissue capture features 116 connected to arms 114 can be deflectable projections that extend between respective ones of arms 114 and the tissue wall to be compressed back toward the respective arm 114 when shunt device 100 is implanted in the tissue wall. Tissue capture features 116 connected to end portions 122 of central flow tube 110 can be secondary arms associated with one of arms 114. Tissue capture features 116 that are a part of arms 114 themselves can be, e.g., a lengthened portion of one of arms 114, separate split arm portions of one of arms 114, and/or interlacing arms 114. Any one or more of tissue capture features 116 can be incorporated alone or in combination on shunt device 100 to aid in anchoring shunt device 100 to the tissue wall and to prevent displacement of shunt device 100.

FIG. 5 is a perspective view of shunt device 100′ including sensor 150′. Shunt device 100′ includes body 102′, which is formed of struts 104′ and openings 106′. Body 102′ includes central flow tube 110′, flow path 112′, arms 114′. Shunt device 100′ also includes and tissue capture features 116′. Central flow tube 110′ has side portions 120′ (including side portion 120A′ and side portion 120B′), end portions 122′ (including end portion 122A′ and end portion 122B′), first axial end 124′, and second axial end 126′. Arms 114′ include distal arms 130′ (including distal arm 130A′ and distal arm 130B′) and proximal arms 132′ (including proximal arm 132A′ and proximal arm 132B′). Distal arms 130′ have terminal ends 134′(including terminal end 134A′ and terminal end 134B′). Proximal arms 132′ have terminal ends 136′ (including terminal end 136A′ and terminal end 136B′). Shunt device 100′ further includes sensor 150′ and sensor attachment portion 152′.

Shunt device 100′ includes a similar structure and design to shunt device 100 described above, except shunt device 100′ additionally includes sensor 150′ connected to sensor attachment portion 152′.

As shown in FIG. 5, sensor 150′ can be attached to shunt device 100′ so that sensor 150′ is positioned in the left atrium when shunt device 100′ is implanted in the tissue wall between the left atrium and the coronary sinus of the heart. Accordingly, sensor 150′ can be attached to one of distal arms 130′. Alternatively, sensor 150′ can be attached to shunt device 100′ so that sensor 150′ is positioned in the coronary sinus when shunt device 100′ is implanted in the tissue wall. In such examples, sensor 150′ can be attached to one of proximal arms 132′. In further examples, an additional sensor can be included on shunt device 100′ to position sensors in both the left atrium and the coronary sinus.

Sensor 150′ is attached to shunt device 100′ at sensor attachment portion 152′. Sensor 150′ can be connected to sensor attachment portion 152′ using any suitable attachment mechanism. For example, sensor 150′ and sensor attachment portion 152′ can include complimentary mating features. Sensor attachment portion 152′ can be an extension of one of arms 114′ of shunt device 100′. In some examples, sensor attachment portion 152′ is an extension of distal arm 130A′. In other examples, sensor attachment portion 152′ is an extension of distal arm 130B′ or one of proximal arms 132′. Alternatively, as shown in FIG. 5, sensor attachment portion 152′ can be a separate split arm portion of one of arms 114′. Sensor attachment portion 152′ can be angled away from a horizontal reference plane (not shown) that is in the plane of the tissue wall adjacent to shunt device 100′ when shunt device 100′ is implanted in the tissue wall. That is, sensor attachment portion 152′ can be angled away from the tissue wall.

Sensor 150′ can be a pressure sensor to sense a pressure in the left atrium. In other examples, sensor 150′ can be any sensor to measure a parameter in the left atrium. In yet other examples, sensor 150′ can be any sensor to measure a parameter in the coronary sinus. Sensor 150′ can include a transducer, control circuitry, and an antenna in one example. The transducer, for example a pressure transducer, is configured to sense a signal from the left atrium. The transducer can communicate the signal to the control circuitry. The control circuitry can process the signal from the transducer or communicate the signal from the transducer to a remote device outside of the body using the antenna. Sensor 150′ can include alternate or additional components in other examples. Further, the components of sensor 150′ can be held in a sensor housing that is hermetically sealed.

### Delivery Catheter 200 (FIGS. 6-7B)

FIG. 6 is a side view of delivery catheter 200. FIG. 7A is a side view of distal portion 214 of delivery catheter 200 in a sheathed state. FIG. 7B is a side view of distal portion 214 of delivery catheter 200 in an unsheathed state. FIGS. 6, 7A, and 7B will be discussed together. FIGS. 6-7B show delivery catheter 200. FIG. 7B shows shunt device 202. Delivery catheter 200 includes proximal end 200A, distal end 200B, proximal portion 210, intermediate portion 212, distal portion 214, handle 216, outer sheath 218, inner sheath 220, bridge 222, nosecone 224, actuation rod 226, side opening 228, and notch 229.

Delivery catheter 200 is one example of a delivery catheter that can be used to implant a shunt device into a patient. Delivery catheter 200 as shown in FIGS. 6-7B is used to implant shunt device 202 (shown in FIG. 7B). Delivery catheter 200 can take other forms in alternate examples. Shunt device 202 can have the structure and design of any suitable shunt device, for example shunt device 100 or 100′ as shown in FIGS. 3A-5. Delivery catheter 200 is shown as being configured to implant shunt device 202 without a sensor in the example shown in FIGS. 6-7B. In alternate examples, delivery catheter 200 can be used to implant a shunt device with a sensor, including any needed modifications to accommodate the sensor.

Delivery catheter 200 includes proximal portion 210 adjacent proximal end 200A of delivery catheter 200, intermediate portion 212 extending from proximal portion 210, and distal portion 214 extending from intermediate portion 212 to distal end 200B of delivery catheter 200. Proximal portion 210 includes handle 216, which can be grasped by a physician to control movement of delivery catheter 200. Handle 216 includes a number of ports through which guide wires, tubes, fluids, or other components or elements may be passed.

Intermediate portion 212 extends outward from handle 216 and is a length of catheter that can be moved through a patient. Outer sheath 218 and inner sheath 220 extend outward from handle 216 and form a portion of intermediate portion 212. Outer sheath 218 covers inner sheath 220.

Distal portion 214 extends from intermediate portion 212. Distal portion 214 includes bridge 222 and nosecone 224. Bridge 222 extends from inner sheath 220 towards nosecone 224. Nosecone 224 extends from bridge 222 to distal end 200B of delivery catheter 200. Bridge 222 is configured to hold shunt device 202. As shown in FIG. 7A, when delivery catheter 200 is in a sheathed state, outer sheath 218 will extend over and cover shunt device 202 on bridge 222. As shown in FIG. 7B, when delivery catheter 200 is in an unsheathed state, outer sheath 218 will be pulled back to expose bridge 222 and shunt device 202 on bridge 222. Nosecone 224 extends outward from bridge 222 and helps guide delivery catheter 200 through a patient's vasculature. Actuation rod 226, also called an actuation arm, extends through a lumen in inner sheath 220 and bridge 222. Actuation rod 226 emerges from side opening 228 in bridge 222 and connects to a first proximal arm of shunt device 202. Side opening 228 extends into a body of bridge 222. Notch 229 extends into the body of bridge 222 opposite side opening 228. Notch 229 is configured to seat a second proximal arm of shunt device 202. The second proximal arm can be retained on bridge 222 prior to deployment by a release wire (not shown) extending through a lumen of bridge 222 and through notch 229.

Delivery catheter 200 will be discussed below in more detail with respect to FIGS. 8A-9R.

### Delivery Method 300 (FIGS. 8A-9R)

FIG. 8A is a flow chart showing steps for creating a puncture in tissue wall TW between coronary sinus CS and left atrium LA. FIG. 8B is a flow chart showing steps for implanting shunt device 202 in tissue wall TW between coronary sinus CS and left atrium LA. FIGS. 9A-9R are schematic views showing the steps for implanting shunt device 202 in tissue wall TW between coronary sinus CS and left atrium LA. FIGS. 8A-9R will be discussed together. FIGS. 8A-8B show method 300. FIG. 8A shows steps 302-316 of method 300. FIG. 8B shows steps 318-334 of method 300.

Step 302 includes advancing guidewire 230 into coronary sinus CS, as shown in FIG. 9A. Guidewire 230 can be inserted using traditional methods. Guidewire 230 is inserted into right atrium RA, through an ostium of coronary sinus CS, and then into coronary sinus CS. Optionally, a catheter having radiopaque markers can be inserted over guidewire 230 and imaging can be done to confirm placement of guidewire 230 in coronary sinus CS. Additionally, contrast can be injected into coronary sinus CS through the catheter to further confirm placement of guidewire 230 in coronary sinus CS. The catheter can then be removed once placement of guidewire 230 in coronary sinus CS is confirmed.

Step 304 includes advancing puncture catheter 232 over guidewire 230 to coronary sinus CS, as shown in FIG. 9B. Puncture catheter 232 is used to puncture tissue wall TW between coronary sinus CS and left atrium LA. Puncture catheter 232 includes catheter body 234 having opening 236 on a first side and balloon 238 on a second side opposite opening 236. Puncture catheter 232 can also include radiopaque markers 239 proximal and distal to opening 236 to confirm placement of puncture catheter 232 in coronary sinus CS. Puncture catheter 232 is advanced into coronary sinus CS so that opening 236 is facing tissue wall TW between coronary sinus CS and left atrium LA. Puncture catheter 232 shown in FIG. 9B is one example of a puncture catheter. In alternate examples, tissue wall TW can be punctured using other puncture catheters or other suitable mechanisms.

Step 306 includes inflating balloon 238 of puncture catheter 232, as shown in FIG. 9C. As balloon 238 is inflated, it will press against coronary sinus CS opposite of tissue wall TW. The inflation of balloon 238 will press puncture catheter 232 against tissue wall TW. Specifically, opening 236 will be pressed against tissue wall TW. Balloon 238 will anchor puncture catheter 232 in position in coronary sinus CS while a puncture is made in tissue wall TW. In alternate examples, any other suitable anchoring mechanism can be used instead of balloon 238. In further examples, step 306 is not needed.

Step 308 includes puncturing tissue wall TW between coronary sinus CS and left atrium LA, as shown in FIG. 9D. Puncture catheter 232 includes puncture arm 240 extending through a lumen in puncture catheter 232. Puncture arm 240 includes sheath 242 and needle 244 positioned in sheath 242 so that it extends out a distal end of puncture sheath 242. Puncture arm 240 can be advanced through puncture catheter 232 and out of opening 236 to puncture through tissue wall TW between coronary sinus CS and left atrium LA.

Puncture catheter 232 should be positioned in coronary sinus CS so that opening 236 of puncture catheter 232 is positioned 2-4 centimeters from the ostium of coronary sinus CS. This will position the puncture through tissue wall TW at the same location. The puncture, and ultimately the placement of shunt device 202 in the puncture, is positioned over the posterior leaflet of mitral valve MV.

Step 310 includes removing needle 244 from puncture catheter 232, as shown in FIG. 9E. Needle 244 can be removed by pulling it proximally through a lumen extending through needle sheath 242 of puncture arm 240. Needle 244 is fully removed from puncture catheter 232, leaving a lumen extending from a proximal end of puncture catheter 232 through a distal end of needle sheath 242.

Step 312 includes advancing guidewire 246 through puncture catheter 232 into left atrium LA, as shown in FIG. 9F. Specifically, guidewire 246 is advanced through a lumen extending through a proximal end of puncture catheter 232 and needle sheath 242 of puncture arm 240. Guidewire 246 is advanced into left atrium LA until it coils in left atrium LA, as shown in FIG. 9F. Once guidewire 246 is fully positioned in left atrium LA, puncture catheter 232 and guidewire 230 can be removed from left atrium LA and coronary sinus CS.

Step 314 includes advancing balloon catheter 248 over guidewire 246 and through the puncture in tissue wall TW, as shown in FIG. 9G. Balloon catheter 248 is advanced through the puncture in tissue wall TW so balloon 250 of balloon catheter 248 is positioned in the puncture in tissue wall TW. Balloon catheter 248 is shown as being a separate device from puncture catheter 232 in the example shown in FIG. 9G. However, in alternate examples, balloon catheter 248 can be inserted through puncture catheter 232 and through the puncture in tissue wall TW.

Step 316 includes inflating balloon 250 of balloon catheter 248 extending through the puncture in tissue wall TW, as shown in FIG. 9H. Balloon 250 extends along a distal portion of balloon catheter 248. As balloon 250 is inflated, it will expand and push open the tissue surrounding the puncture in tissue wall TW. The inflation of balloon 250 will cause the puncture in tissue wall TW to become a wider opening in which a shunt device can be positioned. Balloon 250 can then be deflated and balloon catheter 248 can be removed from left atrium LA and coronary sinus CS.

Step 318 includes advancing delivery catheter 200 over guidewire 246, as shown in FIG. 9I. Delivery catheter 200 has the general structure and design as discussed with reference to FIGS. 6-7B above. Delivery catheter 200 is inserted through coronary sinus CS, through the opening in tissue wall TW, and into left atrium LA. When delivery catheter 200 is properly positioned in tissue wall TW, nosecone 224 will be positioned in left atrium LA, and bridge 222 will extend through tissue wall TW between left atrium LA and coronary sinus CS. Nosecone 224 tapers from a smaller diameter at a distal end to a larger diameter at a proximal end. The taper of nosecone 224 helps to advance nosecone 224 through the opening in tissue wall TW and widens the opening as needed. Bridge 222 holds shunt device 202 (not shown in FIG. 9I) in a collapsed position on bridge 222. Bridge 222 is positioned in tissue wall TW so that shunt device 202 is generally positioned in the opening in tissue wall TW for deployment into the opening.

Step 320 includes withdrawing outer sheath 218 of delivery catheter 200 to release distal arms 252 of shunt device 202, as shown in FIG. 9J. Outer sheath 218 can be withdrawn to expose part of shunt device 202 held on bridge 222 of delivery catheter 200. As outer sheath 218 is withdrawn, distal arms 252 of shunt device 202 will be released and assume their preset shape. Delivery catheter 200 should be positioned in left atrium LA such that when outer sheath 218 is withdrawn to release distal arms 252 of shunts device 202, distal arms 252 of shunt device 202 are positioned in left atrium LA.

Step 322 includes pulling delivery catheter 200 proximally to seat distal arms 252 of shunt device 202 on tissue wall TW, as shown in FIG. 9K. Delivery catheter 200 can be gently pulled proximally to seat distal arms 252 of shunt device 202 on tissue wall TW in left atrium LA. A physician should stop gently pulling on delivery catheter 200 when resistance is sensed, indicating that distal arms 252 have come into contact with tissue wall TW. This will also position a central flow tube of shunt device 202 in the opening in tissue wall TW.

Step 324 includes withdrawing outer sheath 218 of delivery catheter 200 to expose proximal arms 254 of shunt device 202, as shown in FIG. 9L. Outer sheath 218 is withdrawn a set distance to fully expose shunt device 202, including proximal arms 254 of shunt device 202. Delivery catheter 200 should be positioned in left atrium LA, tissue wall TW, and coronary sinus CS so that proximal arms 254 will be positioned in coronary sinus CS when outer sheath 218 is withdrawn. Proximal arms 254 are constrained on bridge 222 of delivery catheter 200 and will not automatically assume their preset shape when outer sheath 218 is withdrawn.

Step 326 includes moving first proximal arm 254A of shunt device 202 towards tissue wall TW using actuation rod 226 of delivery catheter 200, as shown in FIG. 9M. Actuation rod 226 extends through a lumen in delivery catheter 200 and can be actuated forward to move first proximal arm 254A towards tissue wall TW.

Step 328 includes seating first proximal arm 254A on tissue wall TW, as shown in FIG. 9N. Actuation rod 226 of delivery catheter 200 is actuated fully outward to seat first proximal arm 254A on tissue wall TW. When first proximal arm 256A is seated on tissue wall TW, it will be positioned in coronary sinus CS.

Step 330 includes injecting contrast into coronary sinus CS and left atrium LA to confirm placement of shunt device 202 in tissue wall TW, as shown in FIG. 9O. Contrast can be injected through a lumen extending through delivery catheter 200. The contrast can move through coronary sinus CS and left atrium LA. The contrast will highlight shunt device 202 under fluoroscopy to confirm proper placement of distal arms 252 and first proximal arm 254A of shunt device 202 on tissue wall TW.

Step 332 includes removing actuation rod 226 from first proximal arm 254A of shunt device 202, as shown in FIG. 9P. Actuation rod 226 can be held on and removed from first proximal arm 254A using any suitable mechanism. In the example shown in FIG. 9P, a release wire holds actuation rod 226 on first proximal arm 254A. The release wire can be withdrawn proximally to disconnect release wire from first proximal arm 254A. Actuation rod 226 can then be pulled proximally through a lumen of delivery catheter 200 to remove actuation rod 226 from coronary sinus CS.

Step 334 includes withdrawing delivery catheter 200 from coronary sinus CS and left atrium LA to release second proximal arm 254B of shunt device 202, as shown in FIG. 9Q. Second proximal arm 254B is held in place on bridge 222 in notch 229 formed in bridge 222. As delivery catheter 200 is withdrawn, second proximal arm 254B will be released from notch 229 in bridge 222 and take its preset shape. Specifically, second proximal arm 254B will seat upon tissue wall TW as it takes its preset shape. Second proximal arm 245B will be positioned in coronary sinus CS. After second proximal arm 254B is seated on tissue wall TW, shunt device 202 will be fully deployed in tissue wall TW, as shown in FIG. 9R. Delivery catheter 200 and guidewire 246 can then be removed from left atrium LA and coronary sinus CS.

Method 300 is one example of a method that can be used to implant shunt device 202 in tissue wall TW between left atrium LA and coronary sinus CS. Method 300 can include fewer, more, or different steps in alternate examples. Further, puncture catheter 232 and delivery catheter 200 are shown as being separate catheters in the example shown in FIGS. 9A-9R, but can be a single catheter in alternate examples.

### Delivery Device 1000 (FIGS. 10A-12)

FIG. 10A is a perspective view of delivery device 1000 including outer sheath 1002. FIG. 10B is a perspective view of delivery device 1000 with outer sheath 1002 pulled back to expose shunt device 100′. FIGS. 10A-10B will be discussed together. Delivery device 1000 includes outer sheath 1002 (including proximal end 1002A (not shown in FIGS. 10A-10B) and distal end 1002B), straight section 1004, pre-curved section 1006, distal portion 1101, nosecone 1102, molded bridge 1104 (including proximal end 1104A and distal end 1104B), straight portion 1106, and tapered portion 1108. FIG. 10B further shows shunt device 100′, central flow tube 110′, sensor 150′.

Delivery device 1000 is a catheter device that is configured to be transported into and out of a human body. More specifically, delivery device 1000 is configured to transport shunt device 100′ into a human body and then delivery device 1000 is configured to deploy shunt device 100′ within the human body. In some examples, shunt device 100′ is coupled to delivery device 1000, shunt device 100′ is positioned at least partially within outer sheath 1002, and then delivery device 1000 is translated along a guidewire into a human body to properly position shunt device 100′ within the human body. The various examples, features, and methods of using delivery device 1000 will be described in detail below. Although the following discussion specifically describes shunt device 100′ with sensor 150′ coupled to delivery device 1000 and positioned within delivery device 1000, it is to be understood that the following discussion equally applies to shunt device 100 without a sensor.

Delivery device 1000 includes outer sheath 1002, distal portion 1101, and shunt device 100′ including sensor 150′ coupled to delivery device 1000. Outer sheath 1002 can include proximal end 1002A, distal end 1002B, straight section 1004, and pre-curved section 1006. As shown in FIG. 10B, distal portion 1101 can include nosecone 1102 and molded bridge 1104. Nosecone 1102 includes straight portion 1106 and tapered portion 1108. Molded bridge 1104 includes proximal end 1104A and distal end 1104B.

Straight portion 1106 of nosecone 1102 is positioned adjacent and closest to a distal end of delivery device 1000, compared to any other feature/component of delivery device 1000. Straight portion 1106 includes a generally constant diameter extending a length of straight portion 1106, and straight portion 1106 includes an aperture extending fully through straight portion 1106. In some examples, a proximal end of straight portion 1106 can be coupled to a distal end of tapered portion 1108. In alternate examples, straight portion 1106 and tapered portion 1108 can be formed as a unitary, single-piece component. In some examples, straight portion 1106 and tapered portion 1108 can each be constructed from one or more of a thermoplastic polymer, a nylon, or other polymer based material. Tapered portion 1108 is positioned between and coupled to both straight portion 1106 and molded bridge 1104. Tapered portion 1108 of nosecone 1102 also includes an aperture extending fully through tapered portion 1108. The aperture extending through tapered portion 1108 is axially aligned with the aperture extending through straight portion 1106 of nosecone 1102. Further, the apertures extending through straight portion 1106 and tapered portion 1108 are configured to accept a guidewire, discussed with respect to FIGS. 11-12 below.

Molded bridge 1104 includes proximal end 1104A and distal end 1104B. Distal end 1104B of molded bridge 1104 is coupled to an end of tapered portion 1108 of nosecone 1102. In some examples, distal end 1104B of molded bridge 1104 coupled to an end of tapered portion 1108 includes an outer diameter equal to an outer diameter of the end of tapered portion 1108 coupled to distal end 1104B of molded bridge 1104. In other examples, distal end 1104B of molded bridge 1104 may not have the same outer diameter as then end of tapered portion 1108 coupled to molded bridge 1104. As shown, shunt device 100′ surrounds at least a portion of delivery device 1000 and shunt device 100′ is configured to be coupled to delivery device 1000 for delivery into a human body. Further, the features and components of delivery device 1000 are configured to release shunt device 100′ from delivery device 1000 and allow shunt device 100′ to couple to tissue within the human body. To avoid any adverse effects of inserting delivery device 1000 and shunt device 100′ into a human body, delivery device 1000 and shunt device 100′ can each be sterilized before entering the human body.

In the example shown, central flow tube 110′ of shunt device 100′ surrounds a portion of molded bridge 1104, such that a portion of molded bridge 1104 extends through central flow tube 110′. Further, a portion of central flow tube 110′ is positioned adjacent and abutting a portion of molded bridge 1104. The portion of central flow tube 110′ abutting molded bridge 1104 and an arm of shunt device 100′ are coupled to molded bridge 1104 for delivery into the human body. Sensor 150′ is also positioned adjacent and abutting a portion of molded bridge 1104. More specifically, sensor 150′ extends from an arm of shunt device 100′ in a direction towards nosecone 1102 of delivery device 1000. Sensor 150′ is positioned at least partially within a pocket of molded bridge 1104. Shunt device 100′ including sensor 150′ is coupled to delivery device 1000 for translation and placement of shunt device 100′ and sensor 150′ within the human body. Shunt device 100′ can be uncoupled from molded bridge 1104 of delivery device 1000 to allow shunt device 100′ to release from delivery device 1000 and couple to tissue within the human body. After shunt device 100′ and sensor 150′ have been properly placed and coupled to tissue within the human body, delivery device 1000 can be translated out of the human body to remove delivery device 1000 from the human body.

Referring to FIG. 10A, outer sheath 1002 of delivery device 1000 is configured to surround and cover at least a portion of delivery device 1000, such that nosecone 1102 and distal end 1104B of molded bridge 1104 are positioned outside of outer sheath 1002. Further, outer sheath 1002 is configured to fully surround and cover shunt device 100′. As such, outer sheath 1002 is configured to surround and cover shunt device 100′ and a portion of molded bridge 1104 during the insertion of delivery device 1000 into the human body. Further, distal end 1002B of outer sheath 1002 abuts molded bridge 1104 adjacent distal end 1104B of molded bridge 1104 to ensure shunt device 100′ and a portion of molded bridge 1104 remain covered during the insertion and translation into the human body. Covering shunt device 100′ and a portion of molded bridge 1104 with outer sheath 1002 facilitates a smooth insertion of delivery device 1000 into the human body by providing a smooth outer surface for translation into the human body. In some examples, distal end 1002B of outer sheath 1002 can be coupled to distal end 1104B of molded bridge 1104 through a friction fit (otherwise known as an interference fit), such that the frictional force between outer sheath 1002 and molded bridge 1104 holds the components together. In other examples, distal end 1002B of outer sheath 1002 can be coupled to distal end 1104B of molded bridge 1104 through a non-permanent coupling mechanism.

As shown in FIG. 10B, after delivery device 1000 has been translated into the human body and positioned in the correct location, outer sheath 1002 can be pulled in a direction away from nosecone 1102 and molded bridge 1104 to reveal shunt device 100′ and the portion of molded bridge 1104 previously covered by outer sheath 1002. More specifically, outer sheath 1002 is pulled in a direction away from distal end 1104B of molded bridge 1104 and towards proximal end 1104A of molded bridge 1104 to uncouple distal end 1002B of outer sheath 1002 from distal end 1104B of molded bridge 1104. Uncoupling outer sheath 1002 from molded bridge 1104 and pulling outer sheath 1002 in a direction towards proximal end 1002A (not shown in FIGS. 4A-4B) of outer sheath 1002 is the beginning of the process to couple shunt device 100′ to tissue within the human body. Specifically, after outer sheath 1002 is pulled far enough towards proximal end 1002A of outer sheath 1002, at least one arm of shunt device 100′ is allowed to unfold and clamp onto tissue within the human body. Delivery device 1000 proceeds to release the other arms of shunt device 100′ to allow shunt device 100′ and sensor 150′ to fixedly couple to tissue within the human, the steps of which are generally discussed above with respect to FIGS. 8A-9R. An issue with previous sheaths is that the sheaths can kink during insertion and/or induce excess forces and stresses to the tissue of the human body during the insertion process, which is undesirable during a medical procedure. Outer sheath 1002 of the present application includes many advantages over previous sheaths, as will be realized by those skilled in the art.

FIG. 11 is a side view of outer sheath 1002 used in delivery device 1000. FIG. 12 is a side view of outer sheath 1002 in a straight configuration. FIGS. 11-12 will be discussed together. Delivery device 1000 includes outer sheath 1002 (including proximal end 1002A and distal end 1002B), straight section 1004, pre-curved section 1006, first length 1008, second length 1010, third length 1012, fourth length 1014, and inner surface 1016.

Outer sheath 1002 can include proximal end 1002A, distal end 1002B, straight section 1004, and pre-curved section 1006. As shown in FIG. 12, outer sheath 1002 can include first length 1008, second length 1010, third length 1012, and fourth length 1014. As discussed, outer sheath 1002 is a component of delivery device 1000 that is configured to aid in transporting and placing shunt device 100′ in a human body. Further, outer sheath 1002 is kink resistant. That is, outer sheath 1002 is configured to prevent or minimize kinking and undesirable forces and stresses during the insertion of delivery device 1000 into the human body.

Outer sheath 1002 includes proximal end 1002A and distal end 1002B positioned at an opposite end of outer sheath 1002. Proximal end 1002A is the end of outer sheath 1002 positioned closest to a doctor or user's hand during the insertion of delivery device 1000 into the human body. Distal end 1002B is the end of outer sheath 1002 positioned furthest from a doctor or user's hand during the insertion of delivery device 1000 into the human body. In addition, distal end 1002B is the end of outer sheath 1002 that is configured to cover and surround shunt device 100′ and a portion of molded bridge 1004 (shown in FIGS. 10A-10B) during the insertion of delivery device 1000 into the human body. Outer sheath 1002 includes straight section 1004 and pre-curved section 1006 extending from an end of straight section 1004. In the example shown, straight section 1004 extends from proximal end 1002A of outer sheath 1002 in a straight configuration in a direction generally towards distal end 1002B of outer sheath 1002. Further, in the example shown, pre-curved section 1006 extends from a distal end of straight section 1004 in a curved or arched configuration such that together straight section 1004 and pre-curved section 1006 form a hook-shaped outer sheath 1002 in a relaxed configuration.

Straight section 1004 includes an aperture extending fully through straight section 1004. Likewise, pre-curved section 1006 includes an aperture extending fully through pre-curved section 1006. More specifically, outer sheath 1002 has a circular or oval outer cross-section with an aperture extending fully through straight section 1004 and pre-curved section 1006. Therefore, outer sheath 1002 has a generally tubular or cylindrical shape with straight section 1004 having a generally straight hollow cylindrical shape and pre-curved section 1006 having a generally curved hollow cylindrical shape. Outer sheath 1002 fully surrounds and covers components of delivery device 1000 such that the components of delivery device 1000 are positioned within the aperture extending through outer sheath 1002. More specifically, the interior of outer sheath 1002 is hollow to allow shunt device 100′ and a portion of delivery device 1000 to be inserted within the hollow center of outer sheath 1002 for delivery into the human body.

As shown best in FIG. 11, pre-curved section 1006 is a portion of outer sheath 1002 that remains in a curved configuration when in the relaxed position. The curved configuration of pre-curved section 1006 aids in preventing or minimizing kinking and undesirable forces and stresses during the insertion of delivery device 1000 into the human body, discussed further below. In some examples, pre-curved section 1006 can include an inner diameter ranging between 2.40 inches (6.096 centimeters) and 3.20 inches (8.128 centimeters), measuring from axis AA of pre-curved section 1006 to inner surface 1016 of pre-curved section 1006. In some examples, pre-curved section 1006 can include an inner diameter ranging between 2.0 inches (5.08 centimeters) and 4.0 inches (10.16 centimeters), measuring from axis AA of pre-curved section 1006 to inner surface 1016 of pre-curved section 1006. Further, in one specific example, pre-curved section 1006 can include an inner diameter of 2.8 inches (7.112 centimeters), measuring from axis AA of pre-curved section 1006 to inner surface 1016 of pre-curved section 1006. In some examples, pre-curved section 1006 extends from an end of straight section 1004 about axis AA of pre-curved section 1006 at least 180 degrees from the connection point of pre-curved section 1006 and straight section 1004. In another example, pre-curved section 1006 extends from an end of straight section 1004 about axis AA of pre-curved section 1006 between a range of 170 degrees and 210 degrees from the connection point of pre-curved section 1006 and straight section 1004. In other examples, pre-curved section 1006 extends from an end of straight section 1004 about axis AA of pre-curved section 1006 between a range of 130 degrees and 240 degrees from the connection point of pre-curved section 1006 and straight section 1004. In yet other examples, pre-curved section 1006 extends from an end of straight section 1004 about axis AA of pre-curved section 1006 190 degrees from the connection point of pre-curved section 1006 and straight section 1004.

Pre-curved section 1006 is configured to prevent or minimize kinking and undesirable forces and stresses during the insertion of delivery device 1000 into the human body. Specifically, as outer sheath 1002 and delivery device 1000 are translated along a guidewire into the human body, pre-curved section 1006 of outer sheath 1002 will bend into a straight configuration along straight portions of the guidewire and pre-curved section 1006 will bend back into its original curved shape along curved portions of the guidewire. Pre-curved section 1006 having a curved-bias shape allows pre-curved section 1006, and outer sheath 1002 as a whole, to more easily follow the curved portions of guidewire, which prevents or minimizes kinking and undesirable forces and stresses during the insertion of outer sheath 1002 into the human body. As such, during insertion of outer sheath 1002 into the human body, the shape of outer sheath 1002 will vary depending on the tracking shape of sections of the guidewire. Specifically, outer sheath 1002 can enter a straight or generally straight configuration along straight portions of the guidewire and outer sheath 1002 can revert back to a curved configuration along curved portions of the guidewire.

In addition, to prevent or minimize kinking and undesirable forces and stresses during the insertion of delivery device 1000 into the human body, outer sheath 1002 can have a varying stiffness along a length of outer sheath 1002. Specifically, straight section 1004 and pre-curved section 1006 can each include a varying stiffness along a length of each of straight section 1004 and pre-curved section, respectively. In some examples, straight section 1004 of outer sheath 1002 can be stiffer than pre-curved section 1006 of outer sheath 1002. In other examples, a portion near proximal end 1002A of outer sheath 1002 can be stiffer than a portion near distal end 1002B of outer sheath 1002, which can aid in a doctor or user in pushing and pulling on proximal end 1002A of outer sheath 1002 to insert or remove, respectively, outer sheath 1002 from a human body. Further, if a portion near proximal end 1002A of outer sheath 1002 were too soft, it would prevent or increase the difficulty of inserting and removing outer sheath 1002 from a human body due to the lack of rigidity and structure of outer sheath 1002. In further examples, a portion near distal end 1002B of outer sheath 1002 can be softer than proximal end 1002A of outer sheath 1002, allowing distal end 1002B of outer sheath 1002 to easily bend and track around curved portions of a guidewire positioned within a human body.

FIG. 12 is a side view of outer sheath 1002 in a straight configuration, illustrating an example outer sheath 1002 including first length 1008, second length 1010, third length 1012, and fourth length 1014 (not shown to scale). Outer sheath 1002 is shown in a straight configuration for illustrative and descriptive purposes only and it is to be understood that outer sheath 1002 includes a generally hook shaped orientation when in a relaxed state. Although the specific example shown in FIG. 12 includes four lengths 1008, 1010, 1012, and 1014, it is to be understood that other examples of outer sheath 1002 can include more or less than four lengths as described. The different lengths 1008, 1010, 1012, and 1014 of outer sheath 1002 each have varying lengths and stiffnesses to prevent or minimize kinking and undesirable forces and stresses during the insertion of outer sheath 1002 and delivery device 1000 into the human body. The specific lengths and stiffnesses of each of lengths 1008, 1010, 1012, and 1014 can be determined using one or more of mathematical calculations, computational analysis, and experimental testing, among other options.

First length 1008 is positioned adjacent and closest to distal end 1002B of outer sheath 1002, and first length 1008 extends from distal end 1002B of outer sheath 1002 in a direction towards proximal end 1002A of outer sheath 1002. Second length 1010 is positioned adjacent first length 1008, and second length 1010 extends from a distal end of first length 1008 towards proximal end 1002A. Third length 1012 is positioned adjacent and closest to proximal end 1002A of outer sheath 1002, and third length 1012 extends from proximal end 1002A of outer sheath 1002 in a direction towards distal end 1002B of outer sheath 1002. Fourth length 1014 is positioned and extends between second length 1010 and third length 1012. In some examples, first length 1008 of outer sheath 1002 can have a stiffness of 35 durometer. Further, in some examples, second length 1010 of outer sheath 1002 can have a stiffness of 45 durometer. In another example, third length 1012 of outer sheath 1002 can have a stiffness of 72 durometer. In yet another example, fourth length 1014 of outer sheath 1002 can have a stiffness of 55 durometer. The specific stiffness of each of lengths 1008, 1010, 1012, and 1014 can be determined using one or more of mathematical calculations, computational analysis, and experimental testing, among other options, to achieve outer sheath 1002 with optimal bending and flexibility characteristics for translating into and out of a human body.

In some examples, as shown, second length 1010 of outer sheath 1002 can be greater than first length 1008 of outer sheath 1002. Further, in some examples, third length 1012 of outer sheath 1002 can be greater than first length 1008 of outer sheath 1002, and third length 1012 of outer sheath 1002 can be greater than second length 1010 of outer sheath 1002. In some examples, third length 1012 of outer sheath 1002 can be greater than a combined length of first length 1008 and second length 1010 of outer sheath 1002. In other examples, fourth length 1014 of outer sheath 1002 can be greater than first length 1008 of outer sheath 1002, and fourth length 1014 of outer sheath 1002 can be greater than second length 1010 of outer sheath 1002. In yet another example, fourth length 1014 of outer sheath 1002 can be greater than a combined length of first length 1008 and second length 1010 of outer sheath 1002. In another examples, fourth length 1014 of outer sheath 1002 can be less than third length 1012 of outer sheath 1002. The specific lengths of each of lengths 1008, 1010, 1012, and 1014 can be determined using one or more of mathematical calculations, computational analysis, and experimental testing, among other options, to achieve outer sheath 1002 with optimal bending and flexibility characteristics for translating into and out of a human body.

As discussed, the shape of pre-curved section 1006 and the lengths and stiffness of each of lengths 1008, 1010, 1012, and 1014 of outer sheath 1002 are optimized to prevent or minimize kinking and undesirable forces and stresses during the insertion of delivery device 1000 into the human body. In addition, outer sheath 1002 can be constructed from a braided material such that both straight section 1004 and pre-curved section 1006 are constructed from the same braided material. The braided material can include a constant weave pattern extending from proximal end 1002A of outer sheath 1002 to distal end 1002B of outer sheath. The braided material including a constant weave pattern can further reduce or prevent kinking of outer sheath 1002 during the insertion and removal of outer sheath 1002 from a human body. In some examples, the braided material can have a constant weave pattern with between 30 and 40 picks per inch (between about 11.811 and about 15.748 picks per centimeter). In one example, the braided material can have a constant weave pattern with 30 picks per inch (about 11.811 picks per centimeter). In other examples, the braided materials can include less than 30 picks per inch or more than 40 picks per inch (less than about 11.811 picks per centimeter or more than about 15.748 picks per centimeter). In some examples, outer sheath 1002 (i.e., the braided material) can be constructed from a stainless steel covered in a thermoplastic elastomer. The thermoplastic elastomer can be, for example, a Pebax® thermoplastic elastomer. In some examples, outer sheath 1002 can also include a liner or lining material. For example, the liner can be formed of a fluoropolymer material, such as a polytetrafluoroethylene (PTFE).

As will be realized by a person skilled in the art, outer sheath 1002 provides many advantages over previous outer sheaths. Specifically, outer sheath 1002 is optimized to prevent or minimize kinking and undesirable forces and stresses during the insertion of outer sheath 1002 and delivery device 1000 into the human body. Outer sheath 1002 aids in the delivery and placement of shunt device 100′ within a human body, while preventing undesirable tissue stress and/or damage during the insertion and removal process. As such, outer sheath 1002 is an improved sheath with reduced kinking characteristics and improved trackability into the human body, improving the medical procedure of transporting and placing shunt device 100′ (or other implantable device) within the human body.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### Discussion of Possible Examples

The following are non-exclusive descriptions of possible examples of the present invention.

An outer sheath for surrounding and covering components of a delivery device includes a straight section including an aperture extending fully through the straight section. The outer sheath further includes a pre-curved section extending distally from an end of the straight section and including an aperture extending fully through the pre-curved section. The straight section and the pre-curved section include a varying stiffness along a length of the straight section and the pre-curved section.

The outer sheath of the preceding paragraph can optionally include, additionally and/or alternatively, any one or more of the following features, configurations, and/or additional components:

The straight section and the pre-curved section can be constructed from a braided material.

The braided material can include between 30 and 40 picks per inch (between about 11.811 and about 15.748 picks per centimeter).

The braided material can include a constant weave pattern extending from a proximal end of the outer sheath to a distal end of the outer sheath.

The braided material can be a stainless steel covered with a thermoplastic elastomer.

The straight section of the outer sheath can be stiffer than the pre-curved section of the outer sheath.

Together the straight section and the pre-curved section can form a hook-shaped outer sheath.

A diameter of an inner surface of the pre-curved section can range between 2.40 inches (6.096 centimeters) and 3.20 inches (8.128 centimeters).

A diameter of an inner surface of the pre-curved section can be 2.8 inches (7.112 centimeters).

The pre-curved section can extend about an axis of the pre-curved section at least 180 degrees from a connection point of the pre-curved section and the straight section.

The pre-curved section can extend about an axis of the pre-curved section between a range of 170 degrees and 210 degrees from a connection point of the pre-curved section and the straight section.

The pre-curved section can extend about an axis of the pre-curved section 190 degrees from a connection point of the pre-curved section and the straight section.

A distal end of the outer sheath can include a first length of the outer sheath having a stiffness of 35 durometer.

A second length of the outer sheath can be positioned adjacent to the first length of the outer sheath, and the second length of the outer sheath can have a stiffness of 45 durometer.

The second length of the outer sheath can be greater than the first length of the outer sheath.

A third length of the outer sheath can be positioned at a proximal end of the outer sheath, and the third length of the outer sheath can have a stiffness of 72 durometer.

The third length of the outer sheath can be greater than the first length of the outer sheath and the second length of the outer sheath.

The third length of the outer sheath can be greater than a combined length of the first length of the outer sheath and the second length of the outer sheath.

A fourth length of the outer sheath can extend between the second length of the outer sheath and the third length of the outer sheath, and the fourth length of the outer sheath can have a stiffness of 55 durometer.

The fourth length of the outer sheath can be greater than the first length of the outer sheath and the second length of the outer sheath.

The fourth length of the outer sheath can be greater than a combined length of the first length of the outer sheath and the second length of the outer sheath.

The fourth length of the outer sheath can be less than the third length of the outer sheath.

The outer sheath can be kink resistant.

The outer sheath can be constructed from a stainless steel covered with a thermoplastic elastomer.

The outer sheath can be sterilized before insertion into a human body.

The delivery device can be configured to deliver a shunt device within a human body.

The straight section of the outer sheath can have a straight hollow cylindrical shape.

The pre-curved section of the outer sheath can have a curved hollow cylindrical shape.

The outer sheath can fully surround and cover components of the delivery device such that the components of the delivery device are positioned within the aperture extending fully through the pre-curved section.

A proximal end of the outer sheath can be stiffer than a distal end of the outer sheath, allowing a user to push and pull on the proximal end of the outer sheath.

A distal end of the outer sheath can be softer than a proximal end of the outer sheath, allowing the distal end of the outer sheath to bend around a curved guidewire.

While the invention has been described with reference to an exemplary example(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular example(s) disclosed, but that the invention will include all examples falling within the scope of the appended claims.

1. An outer sheath for surrounding and covering components of a delivery device, the outer sheath comprising:
a straight section including an aperture extending fully through the straight section; and
a pre-curved section extending distally from an end of the straight section, wherein the pre-curved section includes an aperture extending fully through the pre-curved section;
wherein the straight section and the pre-curved section include a varying stiffness along a length of the straight section and the pre-curved section; and
wherein a distal end of the outer sheath is softer than a proximal end of the outer sheath, allowing the distal end of the outer sheath to bend around a curved guidewire.

2. The outer sheath of claim 1, wherein the straight section and the pre-curved section form a hook-shaped outer sheath in a relaxed state.

3. The outer sheath of claim 1, wherein the outer sheath is kink resistant, and wherein the outer sheath is constructed from a stainless steel covered with a thermoplastic elastomer.

4. The outer sheath of claim 1, wherein the straight section and the pre-curved section are constructed from a braided material having a constant weave pattern extending from a proximal end of the outer sheath to a distal end of the outer sheath.

5. The outer sheath of claim 1, wherein the pre-curved section extends about a central axis with respect to a curvature of the pre-curved section at least 180 degrees from a connection point of the pre-curved section and the straight section.

6. The outer sheath of claim 1, wherein the pre-curved section extends about a central axis with respect to a curvature of the pre-curved section between a range of 170 degrees and 210 degrees from a connection point of the pre-curved section and the straight section.

7. The outer sheath of claim 1, wherein the pre-curved section extends about a central axis with respect to a curvature of the pre-curved section 190 degrees from a connection point of the pre-curved section and the straight section.

8. The outer sheath of claim 1, wherein a diameter of an inner surface of the pre-curved section ranges between 2.40 inches (6.096 centimeters) and 3.20 inches (8.128 centimeters).

9. An outer sheath for surrounding and covering components of a delivery device, the outer sheath comprising:
a straight section including an aperture extending fully through the straight section; and
a pre-curved section extending distally from an end of the straight section, wherein the pre-curved section includes an aperture extending fully through the pre-curved section;
wherein the straight section and the pre-curved section include a varying stiffness along a length of the straight section and the pre-curved section; and
wherein the straight section and the pre-curved section are constructed from a braided material having a constant weave pattern extending from a proximal end of the outer sheath to a distal end of the outer sheath.

10. The outer sheath of claim 9, wherein the braided material includes between 30 and 40 picks per inch (between about 11.811 and about 15.748 picks per centimeter).

11. The outer sheath of claim 9, wherein the braided material is a stainless steel covered with a thermoplastic elastomer.

12. The outer sheath of claim 9, wherein a proximal end of the outer sheath is stiffer than a distal end of the outer sheath, allowing a user to push and pull on the proximal end of the outer sheath, and wherein the distal end of the outer sheath is softer than the proximal end of the outer sheath, allowing the distal end of the outer sheath to bend around a curved guidewire.

13. The outer sheath of claim 9, wherein a distal end of the outer sheath includes a first length of the outer sheath having a stiffness of 35 durometer.

14. The outer sheath of claim 13, wherein a second length of the outer sheath is positioned adjacent to the first length of the outer sheath, wherein the second length of the outer sheath has a stiffness of 45 durometer, and wherein the second length of the outer sheath is greater than the first length of the outer sheath.

15. The outer sheath of claim 14, wherein a third length of the outer sheath is positioned at a proximal end of the outer sheath, wherein the third length of the outer sheath has a stiffness of 72 durometer, and wherein the third length of the outer sheath is greater than the first length of the outer sheath and the second length of the outer sheath.

16. The outer sheath of claim 15, wherein a fourth length of the outer sheath extends between the second length of the outer sheath and the third length of the outer sheath, wherein the fourth length of the outer sheath has a stiffness of 55 durometer, wherein the fourth length of the outer sheath is greater than the first length of the outer sheath and the second length of the outer sheath, and wherein the fourth length of the outer sheath is less than the third length of the outer sheath.

17. A delivery device for transporting and deploying a shunt device within a human body, the delivery device comprising:
an outer sheath including:
a straight section including an aperture extending fully through the straight section; and
a pre-curved section extending distally from an end of the straight section, wherein the pre-curved section includes an aperture extending fully through the pre-curved section;
wherein the straight section and the pre-curved section include a varying stiffness along a length of the straight section and the pre-curved section; and
a distal portion including a nosecone and a molded bridge.

18. The delivery device of claim 17, wherein the straight section of the outer sheath is stiffer than the pre-curved section of the outer sheath.

19. The delivery device of claim 17, wherein together the straight section and the pre-curved section form a hook-shaped outer sheath.

20. The delivery device of claim 17, wherein the straight section and the pre-curved section are constructed from a braided material having a constant weave pattern extending from a proximal end of the outer sheath to a distal end of the outer sheath.
